(19) United States
(12) Patent Application Publication
(10) Pub. No.: US 2003/0114715 A1
(43) Pub. Date: Jun. 19, 2003

(54) Method for producing alkenyl ethers

(76) Inventors: Arnd Boettcher, Frankenthal (DE); Rolf Pinkos, Bad Durkheim (DE); Rudolf Erich Lorenz, Ludwigshafen (DE)

Correspondence Address: KEIL & WEINKAUF, 1350 CONNECTICUT AVENUE, N.W., WASHINGTON, DC, 20036, US

(21) Appl. No.: 10/240,683

(22) PCT Filed: Mar. 29, 2001
(86) PCT No.: PCT/EP01/03588

(30) Foreign Application Priority Data
Apr. 6, 2000 (DE) 100 17 222.9

(51) Int. Cl. C07C 41/08; C07C 43/20

(52) U.S. Cl. 568/630; 568/688

(57) ABSTRACT

Alkenyl ethers are prepared by reacting the corresponding alcohols or phenols with acetylenes in the liquid phase in the presence of basic alkali metal compounds and a cocatalyst comprising compounds of the formula (Ia) and/or (Ib)

R¹O—(CH₂CH₂CH₂CH₂O)_{n—}H (Ia)

R¹O—(CH₂CH₂CH₂CH₂O)_{n—}H², (Ia)

where R¹, R² are, independently of one another, C₁-C₆-alkyl or C₂-C₆-alkenyl, or R¹ and R² together form a butyl unit and n is 1, 2, 3, 4 or 5.

The present invention relates to an improved process for preparing alkenyl ethers by reacting the corresponding alcohols with acetylenes in the liquid phase in the presence of basic alkali metal compounds and a cocatalyst.

Alkenyl ethers are used, inter alia, as monomeric building blocks in polymers or copolymers, in coatings, adhesives, printing inks and in radiation-curing surface coatings. Further areas of application are the preparation of intermediates, fragrances and flavors and also pharmaceutical products.

Vinyl ethers are generally prepared industrially by reacting the corresponding alcohols with ethyne in the presence of basic catalysts (cf. Ullmann's Encyclopedia of Industrial Chemistry, 6th edition, 1999 Electronic Release, Chapter “Vinyl Ethers—Production”). The vinylation can be carried out either in the liquid phase or in the gas phase. The vinylation in the gas phase is carried out using basic heterogeneous catalysts such as KOH on activated carbon or MgO or CaO. In the liquid phase, the strongly exothermic reaction is generally carried out in the presence of alkali metal alkoxide catalysts, especially in the presence of potassium alkoxides of the alcohols used, at from 120 to 180° C. In the vinylation of primary and secondary aliphatic alcohols, the reaction generally proceeds spontaneously. However, tertiary aliphatic alcohols can be vinylated only slowly and incompletely because of their lower reactivity. The vinylation of tertiary alcohols in particular generally forms considerable amounts of undesirable, sometimes nonvolatile by-products. The same applies in principle to the use of phenolic starting materials.

The great decrease in reactivity from primary via secondary to tertiary alcohols has been described for the vinylation in the gas phase in V. A. Sims and J. F. Vitcha, Ind. Eng. Chem. Prod. Res. Dev., Vol. 2, No. 4, 1963, pages 293-296, and for the vinylation in the liquid phase in E. D. Holly, J. Org. Chem., Vol. 24, 1959, pages 1752-1755. As described in Trofimov, Z. Chem., Vol. 26, 1986, No. 2, pages 41-49, the yield in the vinylation of tertiary alcohols is also low in the presence of superbasic media.

Numerous publications have described dilution of the reaction solution by use of various solvents. The addition of solvents makes it possible to vinylate low molecular weight alcohols under a mild pressure, frequently atmospheric pressure. Solvents disclosed as being suitable, for example in SU 481 589 and SU 1 504 969, are relatively high-boiling by-products which are formed in parallel in the vinylation. GB 717 051 teaches the use as solvents of relatively high molecular weight vinyl ethers which can, for example, be synthesized from a relatively high molecular weight alcohol in a preceding vinylation reaction. Monoglycols, oligoglycols and polyglycols of ethylene oxide, propylene oxide or butylene oxide and their ethers have been described as suitable solvents in JP 04 198 144 A2 and JP 04 095 040 A2.

DD-A 298 775 and DD-A 298 776 disclose the synthesis of alkyl vinyl ethers using a solvent, for example an aliphatic, cycloaliphatic or aromatic hydrocarbon or an ether, and in the presence of a monoglycol, oligoglycol or polyglycol of ethylene oxide, propylene oxide or butylene oxide or an 18-crown-6 or dibenzo-18-crown-6 crown ether as cocatalyst.

According to DE-A 1 812 602, the use of monoglycols, oligoglycols and polyglycols of ethylene oxide, propylene oxide or butylene oxide or their ethers as solvent makes it possible for aryl vinyl ethers to be prepared in a continuous reaction, since the formation of deposits is suppressed and the yield of desired product is increased.

WO 91/05756 describes the use of dimethyl ether and tetraethylene glycol as solvent in the vinylation and epoxyvinylation of 1,1,1-tris(hydroxymethyl)ethane.

The amounts of solvents disclosed in the abovementioned publications are frequently more than half of the total mass of alcohol used. According to the present invention, it has been recognized that only an unsatisfactorily low space-time yield is achieved because of the reaction volume required. Furthermore, it has been recognized according to the present invention that the high contents of solvents both in the synthesis and in the subsequent work-up by distillation result in a high energy consumption (e.g. heating energy, cooling energy). According to the present invention, it was also recognized that when monoglycols, oligoglycols and polyglycols are used, a considerable part of the acetylene added is consumed for vinylating their reactive hydroxy groups and is thus no longer available for the actual vinylation reaction. The solvents used constitute relatively large amounts of frequently expensive starting materials which generally cannot be recovered.

It is an object of the present invention to develop a process for preparing alkenyl ethers which does not have the abovementioned disadvantages and makes it possible to prepare alkenyl ethers in a high space-time yield in a simple way. In particular, the formation of by-products should be considerably suppressed while at the same time using concentrated reactants, so that at most only a small amount of nonvolatile residue is formed and the reaction mixture does not become viscous or solidified.

We have found that this object is achieved by a process for preparing alkenyl ethers by reacting the corresponding alcohols or phenols with acetylenes in the liquid phase in the presence of basic alkali metal compounds and a cocatalyst comprising compounds of the formula (Ia) and/or (Ib)

R¹O—(CH₂CH₂CH₂CH₂O)_{n—}H (Ia)

R¹O—(CH₂CH₂CH₂CH₂O)_{n—}H², (Ia)

where R¹, R² are, independently of one another, C₁-C₆-alkyl or C₂-C₆-alkenyl, or R¹ and R² together form a butyl unit and n is 1, 2, 3, 4 or 5.

The process of the present invention makes it possible to obtain alkenyl ethers in high selectivity and high yield from the corresponding alcohols or phenols and acetylenes in the presence of basic alkali metal compounds and an inexpensive cocatalyst which can easily be separated from the reaction mixture again.

An essential aspect of the process of the present invention is the presence of a cocatalyst (Ia) and/or (Ib)

R¹O—(CH₂CH₂CH₂CH₂O)_{n—}H (Ia)

R¹O—(CH₂CH₂CH₂CH₂O)_{n—}H², (Ia)

where R¹, R² are, independently of one another, unbranched or branched C₁-C₆-alkyl, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

or branched or unbranched C₂-C₆-alkenyl having a double bond in any position, for example ethenyl (vinyl), 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-hexenyl, 2-hexenyl, 3-hexenyl, 4-hexenyl and 5-hexenyl;

or R¹ and R² together form a butenyl unit, specifically CH₂CH₂CH₂CH₂,

and n is 1, 2, 3, 4 or 5,

or a mixture thereof.

Examples of cocatalysts (Ia) and/or (Ib) which can be used in the process of the present invention are 4-methoxy-1-butanol, 4-ethoxy-1-butanol, 4-propoxy-1-butanol, 4-butoxy-1-butanol, 1,4-dimethoxybutane, 1,4-diethoxybutane, 1,4-dipropoxybutane, 1,4-dibutoxybutane, 1-ethoxy-4-methoxybutane, 1-propoxy-4-methoxybutane, 1-butoxy-4-methoxybutane, 1-propoxy-4-ethoxybutane, 1-butoxy-4-ethoxybutane, 1-butoxy-4-propoxybutane, 4-vinyloxy-1-butanol, 4-(isopropenyloxy)-1-butanol, 4-propenyloxy-1-butanol, 1,4-divinyloxybutane, 1,4-bis(isopropenyloxy)butane, 1,4-bis(propenyloxy)butane, 1-vinyloxy-4-methoxybutane, 1-vinyloxy-4-ethoxybutane, 1-vinyloxy-4-propoxybutane, 1-(isopropenyloxy)-4-propoxybutane, 1-(propenyloxy)-4-propoxybutane, 4-(4′-methoxy-1′-butoxy)-1-butanol, 4-(4′-ethoxy-1′-butoxy)-1-butanol, 4-(4′-vinyloxy-1′-butoxy)-1-butanol, bis-(4-methoxy-1-butyl) ether, bis-(4-ethoxy-1-butyl) ether, bis-(4-vinyloxy-1-butyl) ether, 10-crown-2, 15-crown-3 and 20-crown-4.

Preference is given to using cocatalysts of the formulae (Ia) and/or (Ib) in which R¹, R² are, independently of one another, ethyl or vinyl, for example 4-ethoxy-1-butanol, 1,4-diethoxybutane, 4-vinyloxy-1-butanol, 1,4-divinyloxybutane, 1-vinyloxy-4-ethoxybutane, 4-(4′-ethoxy-1′-butoxy)-1-butanol, 4-(4′-vinyloxy-1′-butoxy)-1-butanol, bis-(4-ethoxy-1-butyl) ether and bis-(4-vinyloxy-1-butyl) ether or mixtures thereof.

Particular preference is given to using 4-ethoxy-1-butanol, 1,4-diethoxybutane, 4-vinyloxy-1-butanol, 1,4-divinyloxybutane, 1-vinyloxy-4-ethoxybutane or mixtures thereof. Very particular preference is given to using 1,4-diethoxybutane, 1,4-divinyloxybutane or a mixture thereof.

The cocatalysts used in the process of the present invention can be obtained by means of the following syntheses:

a) 4-Alkenyloxy-1-butanols and 1,4-dialkenyloxybutanes are formed by reacting 1,4-butanediol with acetylenes in the presence of a basic catalyst and are separated by distillation. Thus, 4-vinyloxy-1-butanol and 1,4-divinyloxybutane can be obtained, for example, by reaction of 1,4-butanediol with ethyne and work-up by distillation.

b) 4-Alkoxy-1-butanols and 1,4-dialkoxybutanes are prepared by catalytic hydrogenation of the 4-alkenyloxy-1-butanols and 1,4-dialkenyloxybutanes prepared as described in (a). Suitable hydrogenation catalysts are known to those skilled in the art. It is possible to use, for example, noble metal powder, sponge or black, supported hydrogenation metals such as noble metals, nickel or copper on activated carbon or oxidic support materials or Raney catalysts, e.g. Raney nickel. 1,4-Diethoxybutane can thus be obtained by hydrogenation of 1,4-divinyloxybutane.

As an alternative, the 4-alkoxy-1-butanols and 1,4-dialkoxybutanes can be obtained by etherification of 1,4-butanediol with the corresponding alkanols using etherification methods known to those skilled in the art.

c) 1-Alkenyloxy-4-alkoxybutanes are obtained by reacting the 4-alkoxy-1-butanols obtained as described in (b) with acetylenes as described in (a).

d) Alkoxy and alkenyloxy derivatives of dibutylene glycol and tributylene glycol can be obtained by intermolecular etherification of 1,4-butanediol and subsequent formation of derivatives as described in (a) to (c).

e) 1,4-Butanediol crown ethers can be obtained by intermolecular etherification of 1,4-butanediol.

The cocatalyst to be used according to the present invention is advantageously employed in an amount of from 0.1 to 10% by weight, based on the alcohol or phenol used. Particular preference is given to an amount of from 0.5 to 5% by weight.

Alcohols which can be used as starting materials in the process of the present invention are all unbranched and branched, noncyclic and cyclic, saturated and unsaturated, aliphatic and aromatic alcohols having from 1 to 22 carbon atoms and bearing at least one hydroxy group bound to a nonaromatic carbon, and derivatives thereof.

Examples of aliphatic, noncyclic alcohols are methanol, ethanol, 1-propanol, 2-propanol, 1-butanol, 2-butanol (sec-butanol), 2-methyl-1-propanol (isobutanol), 1-methyl-2-propanol (tert-butanol), 1-pentanol, 2-pentanol, 3-pentanol, 2-methyl-1-butanol, 3-methyl-1-butanol (isoamyl alcohol), 2,2-dimethyl-1-propanol, 2-methyl-2-butanol, 3-methyl-2-butanol, 1-hexanol, 2-hexanol, 3-hexanol, 3,3-dimethyl-3-butanol, 4-methyl-2-pentanol, 2-ethyl-1-butanol, cis-3-hexen-1-ol, 5-hexen-1-ol, 1-heptanol, 2-heptanol, 3-heptanol, 2,4-dimethyl-3-pentanol, 1-octanol, 2-octanol, 3-octanol, 2-ethyl-1-hexanol, 2,4,4-trimethyl-1-pentanol, 1-nonanol, 2-nonanol, 3-nonanol, 4-nonanol, 5-nonanol, 1-decanol, 2,2-dimethyl-1-octanol, 1-dodecanol, 1-tetradecanol (myristyl alcohol), 1-hexadecanol (cetyl alcohol), 1-octadecanol (stearyl alcohol), cis-9-octadecen-1-ol (oleyl alcohol), cis,cis-9,12-octadecadien-1-ol, cis,cis, cis-9,12,15-octadecatrien-1-ol, 1-eicosanol (arachyl alcohol), 1-docosanol (behenyl alcohol).

Examples of aliphatic, cyclic alcohols are cyclopropanol, cyclopropylmethanol, cyclopropylethanol, cyclobutanol, cyclobutylmethanol, cyclobutylethanol, cyclopentanol, cyclopentylmethanol, cyclopentylethanol, 1-methylcyclopentanol, 2-methylcyclopentanol, 3-methylcyclopentanol, cyclohexanol, cyclohexylmethanol, cyclohexylethanol, 1-methylcyclohexanol, 2-methylcyclohexanol, 3-methylcyclohexanol, 4-methylcyclohexanol, cycloheptanol, cyclooctanol, cyclodecanol.

Examples of aromatic alcohols are benzyl alcohol, hydroxydiphenylmethane, 1-phenylethanol, 2-phenylethanol, 2,2-diphenylethanol, 2,2,2-triphenylethanol, 1-naphthyl alcohol, 2-naphthyl alcohol.

Examples of alcohols bearing a plurality of hydroxy groups are. 1,2-ethanediol, 1,2-propanediol, 1,3-propanediol, 1,2-butanediol, 1,4-butanediol, 1,5-pentanediol, 1,6-hexanediol, diethylene glycol, triethylene glycol, tetraethylene glycol, 1,2,3-propanetriol (glycerol), 2-methyl-1,2,3-propanetriol.

Phenols which can be used as starting materials in the process of the present invention are all compounds having from 1 to 12 carbon atoms and their derivatives which have at least one hydroxy group bound to an aromatic carbon.

Examples of phenols are phenol, 2-methylphenol (o-cresol), 3-methylphenol (m-cresol), 4-methylphenol (p-cresol), 2-ethylphenol, 3-ethylphenol, 4-ethylphenol, 2,3-dimethylphenol, 2,4-dimethylphenol, 2,5-dimethylphenol, 2,6-dimethylphenol, 3,4-dimethylphenol, 3,5-dimethylphenol, 1-naphthol, 2-naphthol.

It is naturally also possible to use compounds which have both alcoholic and phenolic hydroxy groups, for example 2-(4′-hydroxyphenyl)ethanol.

In the process of the present invention, preference is given to using aliphatic alcohols. Particular preference is given to noncyclic, saturated, aliphatic alcohols having from 1 to 6 carbon atoms, in particular from 3 to 5 carbon atoms, for example 1-methyl-2-propanol (tert-butanol) and 3-methyl-1-butanol (isoamyl alcohol).

Acetylenes used in the process of the present invention are preferably unbranched and branched alkynes having from 2 to 6 carbon atoms and a terminal triple bond, for example ethyne, propyne, 1-butyne, 1-pentyne, 1-hexyne. Particular preference is given to using ethyne and propyne, in particular ethyne.

Basic alkali metal compounds, also referred to as catalysts, which can be used in the process of the present invention are alkoxides and/or phenoxides of lithium, sodium, potassium, rubidium and/or cesium and mixtures thereof. Preference is given to compounds of sodium and of potassium.

The basic alkali metal compounds are generally used in an amount of from 0.02 to 12%, preferably from 0.05 to 10%, of the molar amount of alcohol or phenol used.

It is generally advantageous to use the alkali metal alkoxides or phenoxides of the alcohols or phenols used in the alkenylation, since this avoids introduction of further extraneous materials which would have to be removed from the reaction system in a preceding step or would reduce the yield of desired product. The appropriate alkali metal compounds can be prepared as required by known methods. Examples of suitable methods are reaction of the corresponding alcohols or phenols with (i) elemental alkali metals, (ii) with hydroxides and removal of the water of reaction formed and (iii) with “foreign” alkoxides or phenoxides and removal of the foreign alcohols or phenols formed.

However, in certain cases it is advantageous to add foreign alkoxides or phenoxides deliberately, for example in cases in which the alkoxides or phenoxides of the alcohols or phenols used are not available and a preceding, separate synthesis is not an option. Preference is given to using low molecular weight compounds, since these have boiling points significantly different from those of the alcohols or phenols used and can easily be separated off by distillation. Among low molecular weight alkoxides, preference is given to using the methoxides, ethoxides, propoxides and 2-propoxides of sodium and potassium. The foreign alkoxides or phenoxides added are likewise alkenylated in this variant and subsequently have to be separated from the desired product. As an alternative, the foreign alcohols or phenols formed in the reaction of the foreign alkoxides and phenoxides with the alcohols or phenols can also be separated off prior to the alkenylation, for example by distillation.

If alkali metal hydroxides are used as starting materials for the basic alkali metal compounds, the water of reaction formed has to be removed prior to the alkenylation. Suitable methods are, for example, distilling off the water, adsorption of the water on suitable desiccants or removal of the water by means of a suitable membrane. It is advantageous to set a residual water content of less than 1% by weight, preferably less than 0.5% by weight, particularly preferably less than 0.2% by weight, based on the total amount of liquid. The appropriate methods are known to those skilled in the art. In this variant, the use of sodium hydroxide and/or potassium hydroxide is preferred.

The process of the present invention can be carried out in the resence of a suitable solvent. The use of a solvent is advantageous or may even be necessary for the success of the rocess in the case of, for example, viscous or solid starting materials. Suitable solvents are ones which are inert under the reaction conditions and can be separated without problems from the desired products, for example by distillation. Examples of suitable solvents are N-methylpyrrolidone, tetrahydrofuran or dialkyl ethers of glycols, diglycols, oligoglycols or olyglycols. In the case of liquid starting materials and/or liquid reaction products, the process of the present invention is preferably carried out without addition of a solvent.

The order of addition of the alcohol or phenol and the basic alkali metal compound and of the cocatalyst and any solvent is not important for the success of the process. The important thing is that the water content of the reaction solution prior to the alkenylation is below the values indicated above. The acetylene is generally metered in in accordance with the progress of the reaction.

The process of the present invention can be carried out batchwise, semicontinuously or continuously. Preference is given to the semicontinuous or continuous variants. In a semicontinuous process, the solution comprising the alcohol or phenol, the basic alkali metal compound, the cocatalyst and, if used, a solvent is placed in the reaction vessel and the acetylene is metered in in accordance with the progress of the reaction. The product solution is normally taken from the reaction vessel only after the reaction is complete. In a continuous process, the acetylene and the solution comprising the alcohol or phenol, the basic alkali metal compound, the cocatalyst and, if used, a solvent are fed in continuously and the resulting solution of the reaction products is taken off continuously.

The alkenylation is generally carried out at from 100 to 200° C., preferably from 130 to 180° C., particularly preferably from 140 to 170° C. It is generally carried out at an acetylene pressure of less than 5 MPa (50 bar), preferablyiless than 3 MPa (30 bar), very particularly preferably less than 2.4 mPa (24 bar). However, the total pressure of the system can be significantly higher, since the gas atmosphere above the reaction solution can further comprise, for example, inert gases such as nitrogen or noble gases which can be introduced by controlled injection. Thus, a total pressure in the system of, for example, 20 MPa abs (200 bar abs) is readily possible. If relatively high molecular weight acetylenes are used, the autogenous acetylene pressure is very low and can be, for example, significantly below 0.1 MPa (1 bar). In the case of low molecular weight acetylenes such as ethyne, propyne and 1-butyne, an acetylene pressure of greater than 0.1 MPa (1 bar) is generally set. In this way, an economical pace-time yield is achieved. If ethyne is used as acetylene in the alkenylation, the alkenylation is preferably carried out at n acetylene pressure (ethyne pressure) of from 0.5 to 3.0 MPa (5 to 30 bar), particularly preferably from 0.8 to 2.4 MPa (8 to 24 bar) and very particularly preferably from 1.6 to 2.0 MPa (16 to 20 bar).

Reactors which can be used for the alkenylation are in principle the apparatuses for gas/liquid reactions described in the relevant technical literature. To achieve a high space-time yield, intensive mixing of the solution comprising the alcohol or phenol, the basic alkali metal compound, the cocatalyst and, if used, a solvent with the acetylene is important. Nonlimiting examples which may be mentioned are stirred vessels, cascades of stirred vessels, flow tubes (preferably with internals), bubble columns and loop reactors.

The reaction product is worked up by known methods. Preference is given to distillation to give a plurality of fractions. The distillations are preferably carried out at a pressure of less than 0.1 MPa abs (1 bar abs). Particularly preferably, not only the alkenyl ether but also the cocatalysts are obtained as fractions. Depending on the choice of the cocatalysts used according to the present invention, they are separated off in a lower-boiling or higher-boiling fraction before or after the alkenyl ether. Various fractions which can be obtained are, without implying a restriction: cocatalyst (before or after alkenyl. ether), alkenyl ether, unreacted alcohol or unreactied phenol, various intermediate. boilers, low boilers and high boilers. Depending on the intention, these can be obtained. as crude fractions or in high purity. It is also possible to combine a number of fractions. The distillation can be carried out batchwise, semicontinuously or continuously. In addition, it can be carried out in one column, if desired with side offtakes, or in a plurality of columns connected in series. Suitable methods are known to those skilled in the art. The alkenyl ether can, as described, readily be obtained in a purity of over 99% by means of the process of the present invention.

In the process of the present invention, it is in principle possible to recirculate any unreacted alcohol or phenol which has been separated off without further purification measures. For this purpose, it is not necessary to recover the starting material in high purity, so that a crude distilled fraction can also be employed. In this case, the fraction to be recirculated should be largely free of relatively high-boiling by-products in order to reduce the formation of relatively high molecular weight by-products and residues. Since a high conversion of alcohol or phenol is achieved in the process of the present invention, separation and recirculation of the starting material can generally be dispensed with.

In the process of the present invention, it is possible and usually advantageous to recover the cocatalyst and reuse it as cocatalyst, i.e. recycle it. It is not necessary to recover the cocatalysts in high purity, so that it is also possible to employ a crude distilled fraction. However, it is advantageous to separate off the products which have a significantly higher boiling point. Any losses of cocatalyst which occur should be made up by addition of fresh cocatalysts.

The process of the present invention is particularly preferably used for preparing alkyl vinyl ethers, in particular for preparing tert-butyl vinyl ether and isoamyl vinyl ether.

In a general embodiment, the basic alkali metal compound (catalyst) and the cocatalyst are added a little at a time to the liquid alcohol, possibly diluted with solvents, and mixed. When using phenols, the use of solvents is advantageous. The resulting solution is then passed over a zeolitic desiccant and introduced into a stirred vessel. The water of reaction is removed by the presence of the desiccant. The acetylene is passed into the now virtually water-free solution at from 100 to 200° C. with intensive mixing. In the case of the preferred use of ethyne, the ethyne is preferably introduced to a pressure of 2.4 MPa (24 bar). Further acetylene.is introduced to replace that which is consumed. After acetylene absorption ceases, the reaction system is depressurized. The reaction solution is transferred to a distillation column and, after removal of the lower-boiling components, the alkenyl ether is isolated at the top in high purity.

In a further general embodiment of the alkenylation of alcohols, a concentrated solution (i.e. about 80% of the maximum solubility) of the basic alkali metal compound in the alcohol is prepared in a mixing vessel. This solution is continuously fed to a vacuum distillation column and the water of reaction formed is taken off at the top. The water-free solution obtained is continuously taken off from the bottom and admixed with further, water-free alcohol and water-free cocatalyst. At this point, the recirculated streams are also fed in. The feed mixture is then fed into a continuously operating loop reactor. There, the reaction with the acetylene takes place at from 100 to 200° C. In the case of the preferred use of ethyne, the ethyne is preferably introduced to a pressure of 2.4 MPa (24 bar). The reaction solution is taken continuously from the loop reactor and worked up by distillation. The alkenyl ether is isolated as a pure product. Recovered, unreacted alcohol and the cocatalyst which has been separated off are recirculated.

In a third, particularly preferred embodiment, a solution of from 0.05 to 8 mol % of potassium hydroxide in an alcohol is prepared in a mixing vessel and admixed with from 0.1 to 10 mol % of 1,4-diethoxybutane and/or 1,4-divinyloxybutane, based on the molar amount of the alcohol used. This solution is fed continuously to a vacuum distillation column and the water of reaction formed is taken off at the top. The virtually water-free solution is continuously taken off at the bottom and fed to a stirred vessel. There, the semicontinuous reaction with the gaseous ethyne takes place at from 130 to 180° C. and a pressure of from 0.1 to 2.0 MPa (1 to 20 bar). After the reaction is complete, the contents of the reactor are discharged and passed to work-up by distillation. The alkyl vinyl ether is obtained in high purity. The fraction comprising the added cocatalyst can, if desired, be recirculated and reused.

The process of the present invention makes possible the simple preparation of alkenyl ethers in a high space-time yield by reaction of the corresponding alcohols or phenols with acetylenes in the presence of basic alkali metal compounds and a cocatalyst. In particular, the formation of by-products is substantially suppressed while at the same time employing concentrated reactants, so that at most a small amount of nonvolatile residue is formed and the reaction mixture is prevented from becoming viscous or solid. Owing to the high conversion of alcohol or phenol of significantly above 90%, frequently above 95%, separation and recirculation of the starting material can generally be omitted. The process of the present invention also makes it possible to alkenylate tertiary alcohols in high yield.

Compared to the known processes, the process of the present invention employs concentrated reactants, which leads not only to the abovementioned advantage of a high space-time yield but also to a reduction in the reaction volume required and thus also to a reduction in the energy required. In contrast to the previously described solvents, which frequently have free hydroxy groups and thus consume a considerable part of the acetylene added, the cocatalysts used according to the present invention display no or at most little reaction with acetylene.

Compared to the known processes without use of a solvent and cocatalyst, the process of the present invention results in a considerably reduced formation of by-products. Low concentrations of cocatalysts of less than 1% by weight are very effective in the process of the present invention.

The alkenyl ethers can be obtained in high purity in the process of the present invention. After fine distillation, purities of greater than 99.9% are achievable. Since the cocatalysts used can in general also be separated off by distillation, it is possible for them to be recirculated and reused.

### EXAMPLES

### Definitions

The values for conversion, selectivity and yield indicated in the description and the examples are defined by the following equations:

conversion=[m_{before}(R—OH)−m_{after}(R—OH)]/m_{before}(R—OH)

selectivity=m_{after}(alkenyl ether)/[m_{before}(R—OH)−m_{after}(R—OH)]

yield=conversion×selectivity=m_{after}(alkenyl ether)/m_{before}(R—OH).

The masses on which the calculation is based:

m_{before}(R—OH): mass of alcohol or phenol used

m_{after}(R—OH): mass of unreacted alcohol or phenol

m_{after}(alkenyl ether): mass of alkenyl ether formed after pure distillation

were determined from the % by area in the gas chromatogram.

### Experimental Method for Examples 1 to 3

100 ml of tert-butanol were in each case admixed with 8% by weight of potassium tert-butoxide, dissolved by stirring and, if applicable, 2.5% by weight of the cocatalyst were added. The reaction mixture was placed in a 300 ml autoclave and pressurized with nitrogen at room temperature to 0.5 MPa abs (5 bar abs). After heating to 160° C., the autoclave was pressurized with ethyne to 2.0 MPa abs (20 bar abs). Ethyne consumed in the reaction was replaced by continuous injection of further amounts at 2.0 MPa abs (20 bar abs). After 12 hours, the experiment was stopped and the reaction product was distilled. Analysis was carried out by gas chromatography.

### Example 1 (Comparative Example Without Cocatalyst)

Example 1 was carried out without addition of a cocatalyst. The conversion of tert-butanol was 75.9%. The desired product tert-butyl vinyl ether was obtained in a yield of 68.4%. In the work-up by distillation, a nonvolatile residue of 6.4% by weight, based on the mixture discharged from the autoclave, was obtained.

### Example 2 (According to the Present Invention)

In Example 2, 2.5% by weight of 1,4-diethoxybutane were added as cocatalyst. The conversion of tert-butanol was 97.9%. The desired product tert-butyl vinyl ether was obtained in a yield of 90.9%. In the work-up by distillation, a nonvolatile residue of 2.7% by weight, based on the mixture discharged from the autoclave, was obtained.

### Example 3 (According to the Present Invention)

In Example 3, 2.5% by weight of 1,4-divinyloxybutane were added as cocatalyst. The conversion of tert-butanol was 95.4%. The desired product tert-butyl vinyl ether was obtained in a yield of 89.1%. In the work-up by distillation, a nonvolatile residue of 2.2% by weight, based on the mixture discharged from the autoclave, was obtained. Tert-butyl vinyl ether was obtained in a purity of 99.2 GC % by area.

### Example 4 (According to the Present Invention)

In Example 4, 122.8 g of phenol were dissolved in 129.1 g of N-methylpyrrolidone by stirring in a 300 ml autoclave and admixed with 8% by weight of potassium hydroxide and 2.5% by weight of 1,4-diethoxybutane. The water content of the solution was determined as 0.74 GC % by area. The autoclave was then pressurized with nitrogen at room temperature to 0.5 MPa abs (5 bar abs). After heating to 190° C., the autoclave was pressurized with ethyne to 2.0 MPa abs (20 bar abs). Ethyne consumed in the reaction was replaced by continuous injection of further amounts at 2.0 MPa abs (20 bar abs). After 24 hours, the experiment was stopped. Analysis was carried out by gas chromatography. The conversion of phenol was 97.7%. The desired product phenyl vinyl ether was obtained in a yield of 80.5%. In the work-up by distillation, a nonvolatile residue of 19.9% by weight, based on the mixture discharged from the autoclave, was obtained.

A summary of the examples is given in Table 1. Under otherwise identical conditions, by far the lowest yield of 68.4% was obtained in the vinylation of tert-butanol without cocatalyst. The two examples using cocatalyst give a significantly higher yield of 89.1 and 90.9%. Significantly less nonvolatile residue was formed due to the positive influence of the cocatalyst. Owing to the significantly higher conversion and the increased selectivity, a significantly higher space-time yield was also achieved in the presence of the cocatalysts.

TABLE 1

| | Starting | | Conversion | Selectivity | Yield | nonvolatile residue |
|---|---|---|---|---|---|---|
| No. | material | Cocatalyst | [%] | [%] | [%] | [% by weight] |
| 1 | tert-butanol | none | 75.9 | 90.1 | 68.4 | 6.4 |
| | | (Comparative | | | | |
| | | Example) | | | | |
| 2 | tert-butanol | 2.5% by weight of | 97.9 | 92.8 | 90.9 | 2.7 |
| | | 1,4-diethoxybutane | | | | |
| 3 | tert-butanol | 2.5% by weight of | 95.4 | 93.4 | 89.1 | 2.2 |
| | | 1,4-divinyloxybutane | | | | |
| 4 | phenol in | 2.5% by weight of | 97.7 | 82.4 | 80.5 | 19.9 |
| | N-methylpyrrolidone | 1,4-diethoxybutane | | | | |

We claim:

1. A process for preparing alkenyl ethers by reacting the corresponding alcohols selected from the group consisting of methanol, ethanol, 1-propanol, 2-propanol, 1-butanol, 2-butanol, 2-methyl-1-propanol, 1-methyl-2-propanol, 1-pentanol, 2-pentanol, 3-pentanol, 2-methyl-1-butanol, 3-methyl-1-butanol, 2,2-dimethyl-1-propanol, 2-methyl-2-butanol, 3-methyl-2-butanol, 1-hexanol, 2-hexanol, 3-hexanol, 3,3-dimethyl-3-butanol, 4-methyl-2-pentanol, 2-ethyl-1-butanol, cis-3-hexen-1-ol, 5-hexen-1-ol, 1-heptanol, 2-heptanol, 3-heptanol, 2,4-dimethyl-3-pentanol, 1-octanol, 2-octanol, 3-octanol, 2-ethyl-1-hexanol, 2,4,4-trimethyl-1-pentanol, 1-nonanol, 2-nonanol, 3-nonanol, 4-nonanol, 5-nonanol, 1-decanol, 2,2-dimethyl-1-octanol, 1-dodecanol, 1-tetradecanol, 1-hexadecanol, 1-octadecanol, cis-9-octadecen-1-ol, cis,cis-9,12-octadecadien-1-ol, cis,cis,cis-9,12,15-octadecatrien-1-ol, 1-eicosanol, 1-docosanol, cyclopropanol, cyclopropylmethanol, cyclopropylethanol, cyclobutanol, cyclobutylmethanol, cyclobutylethanol, cyclopentanol, cyclopentylmethanol, cyclopentylethanol, 1-methyl-cyclopentanol, 2-methyl-cyclopentanol, 3-methyl-cyclopentanol, cyclohexanol, cyclohexylmethanol, cyclohexylethanol, 1-methyl-cyclohexanol, 2-methyl-cyclohexanol, 3-methyl-cyclohexanol, 4-methyl-cyclohexanol, cycloheptanol, cyclooctanol, cyclodecanol, benzyl alcohol, hydroxydiphenylmethane, 1-phenylethanol, 2-phenylethanol, 2,2-diphenylethanol, 2,2,2-triphenylethanol, 1,2-ethanediol, 1,2-propanediol, 1,3-propanediol, 1,2-butanediol, 1,5-pentanediol, 1,6-hexanediol, diethylene glycol, triethylene glycol, tetraethylene glycol, 1,2,3-propanetriol and 2-methyl-1,2,3-propanetriol or the corresponding phenols with acetylenes in the liquid phase in the presence of basic alkali metal compounds and a cocatalyst comprising compounds of the formula (Ia) and/or (Ib)
R¹O—(CH₂CH₂CH₂CH₂O)_{n—}H (Ia)
R¹O—(CH₂CH₂CH₂CH₂O)_{n—}H², (Ia)
where R¹, R² are, independently of one another, C₁-C₆-alkyl or C₂-C₆-alkenyl, or R¹ and R² together form a butyl unit and n is 1, 2, 3, 4 or 5.

2. A process as claimed in claim 1, wherein the cocatalyst used comprises compounds of the formulae (Ia) and/or (Ib), in which R¹, R² are, independently of one another, ethyl or vinyl.

3. A process as claimed in claim 1 or 2, wherein the cocatalyst used is 1,4-diethoxybutane, 1,4-divinyloxybutane or a mixture thereof.

4. A process as claimed in any of claims 1 to 3, wherein the cocatalyst (Ia) and/or (Ib) is used in an amount of from 0.1 to 10% by weight, based on the alcohol used or the phenol used.

5. A process as claimed in any of claims 1 to 4, wherein the basic alkali metal compounds are used in an amount of from 0.05 to 10% of the molar amount of the alcohol or phenol used.

6. A process as claimed in any of claims 1 to 5, wherein the reaction of the alcohols or phenols with the acetylenes is carried out at from 100 to 200° C. and an acetylene pressure of less than 5 MPa.

7. A process as claimed in any of claims 1 to 6, wherein the cocatalyst is recovered and reused as cocatalyst.

8. A process as claimed in any of claims 1 to 8, wherein aliphatic alcohols are used.

9. A process as claimed in any of claims 1 to 8, wherein alkyl vinyl ethers are prepared.
